(19) United States
(12) Patent Application Publication
(10) Pub. No.: US 2025/0248475 A1
(43) Pub. Date: Aug. 7, 2025

(54) FOOTWEAR ARTICLE

(71) Applicant: Walk With Path Limited, Waltham Abbey Essex (GB)

(72) Inventors: Lise Sperling Pape, Waltham Abbey Essex (GB); Luke Staniczek, Waltham Abbey Essex (GB)

(21) Appl. No.: 18/855,118

(22) PCT Filed: Apr. 4, 2023
(86) PCT No.: PCT/IB2023/053418

§ 371 Date: Oct. 8, 2024

(30) Foreign Application Priority Data
Apr. 8, 2022 (GB) 2205158.5

(51) Int. Cl. A43B 3/44; A61B 5/00; A61B 5/01; A61B 5/103

(52) U.S. Cl. CPC A43B 3/44; A61B 5/01; A61B 5/1038; A61B 5/6807; A61B 5/746; A61B 2560/0214; A61B 2560/0228

(57) ABSTRACT

Aspects of the invention provide an article of footwear comprising: a technical housing comprising at least one sensor for determining a parameter of a user; a cushioned layer; and a locking mechanism configured to selectively lock the cushioned layer to the technical housing, wherein the technical housing comprises a main body formed from a first flexible material and configured to house the at least one sensor, and a protective cover configured to overlay the at least one sensor, wherein the protective cover is formed from a second flexible material, and wherein the cushioned layer is arranged to be positioned on top of the technical housing.

### FIELD

The present invention relates to a footwear article.

### BACKGROUND

The applicant has previously developed hardware implemented as either part of footwear or an attachment to footwear and uses pressure sensors to determine the pressure exerted by a user's foot on a surface. In response visual or tactile feedback, known as cues, are presented to the user to enable the user to overcome freezing of gait. The applicant's hardware solution utilises pressure sensors embedded in an insole and a battery to power the pressure sensors and other components. Details are available in granted UK patent 2538445.

In the age of technology, more and more technical solutions are being implemented into conventional articles such as shoes. This gives rise to the problem of providing power to electronic components in a safe and reliable manner. Such electronic components need to be protected against the weight of the user and accessible to enable charging of batteries, replacement, and repair.

It is against this background that the present invention has arisen.

### SUMMARY

In one aspect of the invention, an article of footwear comprises: a technical housing comprising at least one sensor for determining a parameter of a user; a cushioned layer; and a locking mechanism configured to selectively lock the cushioned layer to the technical housing, wherein the technical housing comprises a main body formed from a first flexible material and configured to house the at least one sensor, and a protective cover configured to overlay the at least one sensor, wherein the protective cover is formed from a second flexible material, and wherein the cushioned layer is arranged to be positioned on top of the technical housing.

The insole may be inserted into a footwear article, i.e., shoes, trainers or boots and removed again as necessary. The technical housing is intended to house electronic circuitry as part of technical functionality that is added to conventional shoes. The cushioned layer provides protection for the electronic circuitry while simultaneously ensuring that the insole provides sufficient comfort for the wearer of the footwear article. The cushioned layer may be selectively removed from the technical housing by releasing the locking mechanism. This allows users to adjust the level of support provided by the insole based on their individual needs and preferences. Furthermore, removal of the cushioned layer provides access to the electronic circuitry housed within the technical housing for cleaning and maintenance. The locking mechanism ensures that the cushioned layer is not inadvertently dislodged during normal use and/or putting on and taking off the footwear article.

Provision of sensors within a footwear article beneficially adds technical functionality to conventional articles of footwear. Articles of footwear can be provided with pressure sensors to determine the phase of gait of a user. Such sensors may simply determine whether a user's foot is on the ground, or not, or may determine a pressure value. Many types of sensors may be incorporated into a footwear article. In the context of the invention, such sensors may be incorporated into the technical housing of an insole to be inserted into a footwear article.

The technical housing may further comprise a power source for providing electrical power to the at least one sensor.

Prior art footwear articles comprise an insole with embedded technology, including batteries. The present invention provides a power source within a technical housing. Removal of the cushioned layer exposes the power source within the technical housing to enable charging or replacement.

The locking mechanism may comprise a projection as part of one of the technical housing or cushioned layers, and a matching indent as part of the other of the technical housing or cushioned layer. The projection and matching indent may together define a snap fit connection.

Engagement of the projection with the indent provides a positive interface between the technical housing and the cushioned layer. This is necessary to ensure that the cushioned layer is not permitted to move relative to the technical layer while the footwear article is being worn. However, it is also necessary for the cushioned layer to be disengaged from the technical housing. This is achieved by deforming the rear part of the insole such that the projection is moved out of engagement with the indent.

The at least one sensor may comprise at least one pressure sensor. The at least one pressure sensor may comprise a pressure sensor array distributed across the technical housing in both the x and y dimensions.

Monitoring of pressure has many advantageous purposes. In one embodiment, a change of pressure over time, or a sustained pressure at one location for an extended period, may indicate that the wearer is at enhanced risk of developing an ulcer or other foot related injury or condition.

Embodiments of the invention may utilise a pressure sensor, or an array of pressure sensors, for a variety of purposes. For example, a single pressure sensor may be configured to determine simply if a user's foot is in contact with a surface, or not. In another example, the actual pressure exerted by a user's foot may be determined. In yet another example, the user's pressure distribution may be determined by the at least one pressure sensor.

Use of pressure sensors advantageously facilitates the recording of data that can be used to monitor the wearer's gait and other parameters of how they walk and run. This data can be used by clinicians/physicians to identify any deficiencies in gait and form an assessment of fall risk under different conditions. Various algorithms, which are outside of the scope of embodiments of the present invention, may be employed for this purpose.

The at least one sensor may further comprise at least one temperature sensor. The at least one temperature sensor may comprise three temperature sensors positioned at the forefoot, mid-sole and heel positions of the technical housing.

The use of temperature sensors in footwear articles is advantageous in identifying diabetic foot ulcers. Individuals suffering from diabetes are at higher risk of developing foot ulcers than the general population due to impaired nerve function and reduced blood flow to the feet. Fluctuation of temperature can indicate inflammation of an individual's foot. Temperature fluctuation may be monitored in terms of a difference between left and right feet, increase/decrease in temperature at a particular location. The presence of inflammation is a warning sign that a diabetic foot ulcer is at risk of developing. Therefore, detection of likely inflammation provides the opportunity for the individual to take remedial action.

The main body of the technical housing may be in the form of a substantially planar base with a perpendicular lip extending upwardly from the perimeter of the base and extending at least partially around the perimeter of the base. The perpendicular lip may be arranged to extend around the perimeter of a rear end of the main body of the technical housing to a point substantially in a region of a midpoint of the main body of the technical housing. The perpendicular lip may be formed from a hard plastic material. The first flexible material may be polyurethane, and the second flexible material may be ethylene vinyl acetate.

Footwear is subjected to significant compression and torsional forces during normal use. Any electronic components, i.e., sensors, therefore, require protecting to protect against damage. Protection of the electronic components needs to be balanced with comfort for the wearer of the footwear. The present invention achieves this by positioning the electronic components within the insole in a position that experience reduced compressional and torsional forces as compared to other positions of the insole. The insole is constructed in such a way that comfort is prioritised at the forefoot position and the need for comfort and protection is balanced at the heel position.

The cushioned layer may comprise a supporting element and a deformable surface element, wherein the locking mechanism part of the cushioned layer is defined by the supporting element.

Provision of a cushioned layer advantageously provides customisation of a footwear article according to embodiments of the invention. The cushioned layer can be replaced to provide differing levels of support and cushioning to suit each individual user. The custom layer may also be replaced in that it becomes damaged or contaminated with bodily fluids. Furthermore, replaceable cushioned layers permit the same footwear article to be used by multiple users in a hygienic manner so that the higher value electronics of the technical layer can be used between patients in a clinical setting, for example. Cushioned layers of varying designs and properties may be developed by third party providers.

A wireless charging coil may be provided within the technical housing or cushioned layer and at least one magnet is positioned adjacent to the wireless charging coil, wherein the wireless charging coil is configured to charge the power source.

Given the nature of aspects of the invention, a simple and reliable means of charging a power source, i.e., a battery, is essential. Aspects of the invention enable the main charging hardware to remain within the insole during use. The insole does not require removing or a cable being plugged into it in order to enable charging of the power source. Instead, a magnetic charger simply needs to be positioned in the vicinity of the wireless charging coil.

In another aspect of the invention, an item of footwear comprises a sole and an upper, wherein the sole is embedded with one or more electronic components and a power source for providing power to the one or more electronic components, the sole further comprising a wireless charging coil and at least one magnet positioned adjacent to the wireless charging coil, wherein the wireless charging coil is configured to charge the power source.

Another aspect of the invention provides a method of calibrating sensors embodied in an item of footwear, the method comprising: receiving information representative of the material properties of a cushioned layer of material overlaying one or more sensors; retrieving a calibration factor for the cushioned layer of material; applying the correction factor to data received from the one or more sensors; recording or presenting readings from the one or more sensor that are adjusted in accordance with the applied correction factor.

Another aspect of the invention provides a method of identifying a foot related condition, the method comprising: receiving data from one or more sensors associated with a first footwear article worn by a user on a first foot; receiving data from one or more sensors associated with a second footwear article worn by the user on a second foot; comparing the data received from the one or more sensors associated with the first footwear article with the data received from the one or more sensors associated with the second footwear article; determining a difference between the data received from the one or more sensors associated and the second footwear article; comparing the determined difference to a threshold; and if the difference exceeds the threshold, generating an alert for presentation to the user and/or for transmitting to a remote electronic device.

### FIGURES

The above and other objects and advantages of the invention of the disclosures will be apparent upon consideration of the following detailed description, taken in conjunction with the accompanying drawings in which:

FIG. 1 shows a plan view of an article of footwear according to aspects and embodiments of the invention.

FIG. 2. is a detailed view of a part of the side of the insole of FIG. 1.

FIG. 3 is an exploded view of the insole of FIGS. 1 to 3.

FIG. 4 illustrates a method of calibrating sensors according to an embodiment of the invention.

FIG. 5 illustrates a method of identifying a foot related invention according to an embodiment of the invention.

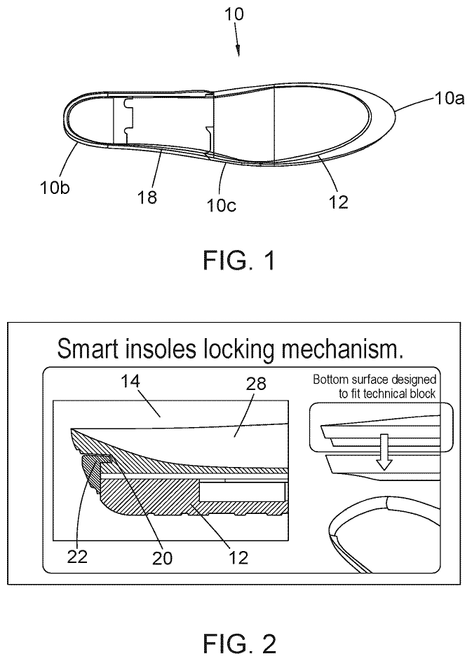

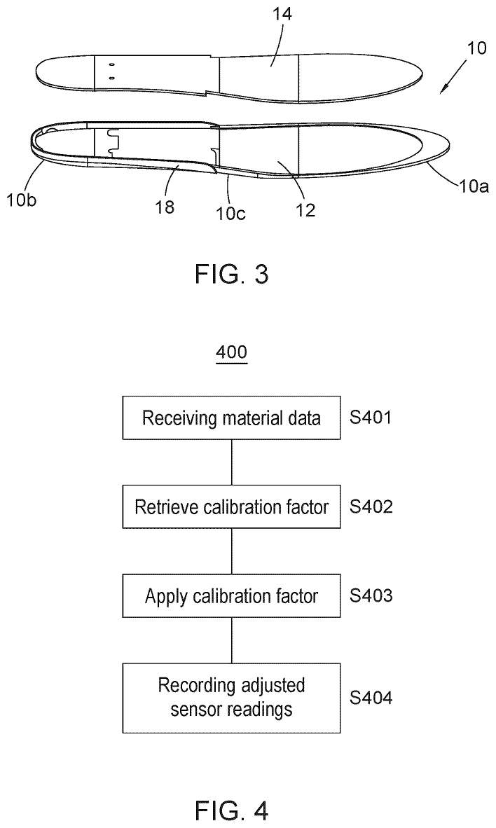

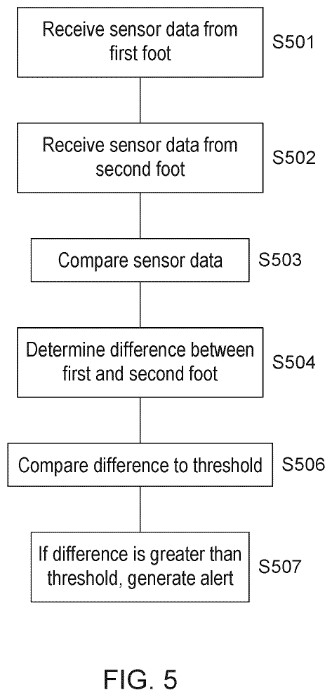

### DESCRIPTION

The following description of the preferred embodiment(s) is merely exemplary in nature and is in no way intended to limit the invention, its application, or uses.

The description of illustrative embodiments according to principles of the present invention is intended to be read in connection with the accompanying drawings, which are to be considered part of the entire written description. In the description of embodiments of the invention disclosed herein, any reference to direction or orientation is merely intended for convenience of description and is not intended in any way to limit the scope of the present invention. Relative terms such as “lower,” “upper,” “horizontal,” “vertical,” “above,” “below,” “up,” “down,” “top” and “bottom” as well as derivatives thereof (e.g., “horizontally,” “downwardly,” “upwardly,” etc.) should be construed to refer to the orientation as then described or as shown in the drawing under discussion. These relative terms are for convenience of description only and do not require that the apparatus be constructed or operated in a particular orientation unless explicitly indicated as such. Terms such as “attached,” “affixed,” “connected,” “coupled,” “interconnected,” and similar refer to a relationship wherein structures are secured or attached to one another either directly or indirectly through intervening structures, as well as both movable or rigid attachments or relationships, unless expressly described otherwise. Moreover, the features and benefits of the invention are illustrated by reference to the exemplified embodiments. Accordingly, the invention expressly should not be limited to such exemplary embodiments illustrating some possible non-limiting combination of features that may exist alone or in other combinations of features; the scope of the invention being defined by the claims appended hereto.

As illustrated in the figures, an insole (10) for a footwear article comprises a technical housing (12) and a cushioned layer (14). The insole (10) extends from a front part (10a), or forefoot, to a rear part (10b), or heel. The main body of the technical housing (12) is formed from a flexible material such as polyurethane and can be cut to size along predefined cutting lines at the front part thereof. The thickness of the technical layer (12) is 2.85 mm from the front part (10a) up to a mid-part (10c) and 6.05 mm from the rear part (10b) up to the mid-part (10c). A rigid lip (18) surrounds the perimeter of the rear part of the technical layer (12) and extends upwardly a distance of 8.35 mm from the base of the technical block (12). The rigid lip (18) therefore projects above the upper surface of the main body of the technical block by a distance of 2.3 mm. The overall thickness of the main body of the technical block (12) includes a protective layer of ethylene-vinyl acetate.

The rigid lip (18) of the technical housing (12) comprises an indent (20) for receiving a matching projection (22) of the cushioned layer (14). This is more particularly shown in FIG. 2. The projection (22) is resiliently biased to engage with the indent (20) so as to form a snap-fit type connection. The projection (22) may be moved out of engagement with the indent (20) to permit the cushioned layer (14) to be removed from the technical housing (12).

The technical housing (12) incorporates electronic circuitry. In one embodiment, the electronic circuitry comprises a pressure sensor array and a plurality of temperature sensors. The pressure sensor array is arranged to extend in x and y dimensions from the front part (10a) to the rear part (10b) of the insole (10). The technical housing may further comprise a power source, i.e., a rechargeable lithium-ion battery, and a controller. The controller may receive data from the pressure sensor array and temperature sensors and process such data to communicate an output signal to one or more external devices or electronic components. The technical housing (12) may further comprise a communications module, i.e., Bluetooth® or ANT+, to enable communication with an external device.

The technical housing (12) may further comprise a wireless charging coil (not shown) around which is positioned at least one magnet. A magnetic wireless charger may engage the magnet incorporated in the technical housing (12) to enable wireless charging of the power source.

The cushioned layer (14) comprises a support element (28) that is at least partially formed from a semi-rigid perimeter and a deformable surface element (30) that provides cushioned support for the wearer's foot. The rearmost part of the support element (28) includes the indent (20).

The electronic circuitry of embodiments of the invention enables data to be collected relating to the way in which a wearer walks and stands. Additional sensors such as an accelerometer, gyroscope, magnetometer, for example, may be incorporated within the electronic circuitry. The combination of sensors incorporated within the electronic circuitry enables a wide array of data to be collected including: pressure, cadence, symmetry of gait, steps, temperature distribution, percentage time each foot is on the floor, walking speed, for example.

In response to the collected data, certain stimulatory responses may be delivered to the wearer's feet. For example, if the wearer is walking in a manner that is imparting significant pressure to one region of the wearer's foot, a vibration stimulation may be delivered to encourage the wearer to shift stance.

The collected data may be transmitted over a wireless or wired connection to an external computing device and/or via the cloud to a web-based dashboard or portal. The dashboard or portal may display the data in raw format and/or may display certain information after processing to indicate a wide variety of measurements and/or scores, for example. In one embodiment, the dashboard or portal may display: number of steps taking over a defined time period, temperature distribution over time, a. graph of cadence, a graph of walking speed, percentage time that both feet are on the floor, for example.

A method (S400) of calibrating sensors embodied in a footwear article is illustrated in FIG. 4. At step S401 information representative of the material properties of a cushioned layer of material overlaying one or more sensors is received. The received information may include: thickness of the cushioned layer of material, thermal properties and firmness. The data may be input manually into a computer software application and may be transmitted to the control circuitry by way of Bluetooth® or WiFi, for example. At step S402, a correction factor is determined based on the received information concerning the cushioned layer of material. The correction factor may either be determined locally at the footwear article or remotely and transmitted to the control circuitry by way of Bluetooth® or WiFi, for example. The correction factor is stored in memory hosted locally at the footwear article at step S403 sensor readings are received by the footwear article from one or more sensors. At step S404 the correction factor is applied to the sensor readings. At step S405 the corrected sensor readings are transmitted to a remote electronic device over Bluetooth® or WiFi, for example.

A method (S600) of identifying a foot related condition is illustrated at FIG. 6. At step S601, data is received from one or more sensors associated with a first footwear article worn by a user on a first foot. At step S602 data is received from one or more sensors associated with a second footwear article worn by the user on a second foot. At step S603 the data received from the first footwear article and second footwear article is compared. At step S604 a difference between the data received from the first footwear article and the second footwear article is determined. At step S605 the determined difference between the data received from the first footwear article and second footwear article is compared to a threshold. At Step S606 if the difference exceeds the threshold an alert is generated for presentation to the user and/or for transmitting to a remote electronic device.

A web portal, or dashboard, may be provided to interface with the footwear article (10) via a communications link over Bluetooth®, WiFi or NFC, for example. The web portal, or dashboard, may be used by a clinician to transfer parameters and data to/from the footwear article (10). Furthermore, data from the footwear article (10) may be transferred to the web portal, or dashboard, and arranged for display in a manner that portrays a wide variety of metrics.

The foregoing disclosure provides illustration and description but is not intended to be exhaustive or to limit the implementations to the precise form disclosed. Modifications may be made considering the above disclosure or may be acquired from practice of the implementations. As used herein, the term “component” is intended to be broadly construed as hardware, firmware, or a combination of hardware and software. It will be apparent that systems and/or methods described herein may be implemented in different forms of hardware, firmware, and/or a combination of hardware and software. The actual specialized control hardware or software code used to implement these systems and/or methods is not limiting of the implementations. Thus, the operation and behavior of the systems and/or methods are described herein without reference to specific software code—it being understood that software and hardware can be used to implement the systems and/or methods based on the description herein. As used herein, satisfying a threshold may, depending on the context, refer to a value being greater than the threshold, greater than or equal to the threshold, less than the threshold, less than or equal to the threshold, equal to the threshold, and/or the like, depending on the context. Although combinations of features are recited in the claims and/or disclosed in the specification, these combinations are not intended to limit the disclosure of various implementations. In fact, many of these features may be combined in ways not specifically recited in the claims and/or disclosed in the specification.

Although each dependent claim listed below may directly depend on only one claim, the disclosure of various implementations includes each dependent claim in combination with every other claim in the claim set. No element, act, or instruction used herein should be construed as critical or essential unless explicitly described as such. Also, as used herein, the articles “a” and “an” are intended to include one or more items and may be used interchangeably with “one or more.” Further, as used herein, the article “the” is intended to include one or more items referenced in connection with the article “the” and may be used interchangeably with “the one or more.” Furthermore, as used herein, the term “set” is intended to include one or more items (e.g., related items, unrelated items, a combination of related and unrelated items, and/or the like), and may be used interchangeably with “one or more.” Where only one item is intended, the phrase “only one” or similar language is used. Also, as used herein, the terms “has,” “have,” “having,” or the like are intended to be open-ended terms. Further, the phrase “based on” is intended to mean “based, at least in part, on” unless explicitly stated otherwise. Also, as used herein, the term “or” is intended to be inclusive when used in a series and may be used interchangeably with “and/or,” unless explicitly stated otherwise (e.g., if used in combination with “either” or “only one of”).

1. An article of footwear comprising: a technical housing comprising at least one sensor for determining a parameter of a user; a cushioned layer; and a locking mechanism configured to selectively lock the cushioned layer to the technical housing, wherein the technical housing comprises a main body formed from a first flexible material and configured to house the at least one sensor, and a protective cover configured to overlay the at least one sensor, wherein the protective cover is formed from a second flexible material, and wherein the cushioned layer is arranged to be positioned on top of the technical housing.

2. An article of footwear according to claim 1, wherein the technical housing further comprises a power source for providing electrical power to the at least one sensor.

3. An article of footwear according to claim 1, wherein the locking mechanism comprises a projection as part of one of the technical housing or cushioned layer and a matching indent as part of the other of the technical housing or cushioned layer.

4. An article of footwear according to claim 3, wherein the projection and matching indent together define a snap fit connection.

5. An article of footwear according to claim 1, wherein the at least one sensor comprises at least one pressure sensor.

6. An article of footwear according to claim 1, wherein the at least one sensor further comprises at least one temperature sensor.

7. An article of footwear according to claim 1, wherein the main body of the technical housing is in the form of a substantially planar base with a perpendicular lip extending upwardly from the perimeter of the base and extending at least partially around the perimeter of the base.

8. An article of footwear according to claim 7, wherein the perpendicular lip is arranged to extend around the perimeter of a rear end of the main body of the technical housing to a point substantially in a region of a midpoint of the main body of the technical housing.

9. An article of footwear according to claim 7, wherein the perpendicular lip is formed from a hard plastic material.

10. An article of footwear according to claim 1, wherein the first flexible material is polyurethane, and the second flexible material is ethylene vinyl acetate.

11. An article of footwear according to claim 5, wherein the at least one pressure sensor comprises a pressure sensor array distributed across the technical housing in both the x and y dimensions.

12. An article of footwear according to claim 6, wherein the at least one temperature sensor comprises three temperature sensors positioned at the forefoot, mid-sole, and heel positions of the technical housing.

13. An article of footwear according to claim 1, wherein the cushioned layer comprises a supporting element and a deformable surface element, wherein the locking mechanism part of the cushioned layer is defined by the supporting element.

14. An article of footwear according to claim 1, wherein a wireless charging coil is provided within the technical housing or cushioned layer and at least one magnet is positioned adjacent to the wireless charging coil, wherein the wireless charging coil is configured to charge the power source.

15. (canceled)

16. A method of calibrating sensors embodied in an item of footwear, the method comprising:
receiving information representative of the material properties of a cushioned layer of material overlaying one or more sensors;
retrieving a calibration factor for the cushioned layer of material;
applying the correction factor to data received from the one or more sensors; recording or presenting readings from the one or more sensor that are adjusted in accordance with the applied correction factor.

17. A method of identifying a foot related condition, the method comprising:
receiving data from one or more sensors associated with a first footwear article worn by a user on a first foot;
receiving data from one or more sensors associated with a second footwear article worn by the user on a second foot;
comparing the data received from the one or more sensors associated with the first footwear article with the data received from the one or more sensors associated with the second footwear article;
determining a difference between the data received from the one or more sensors associated and the second footwear article; comparing the determined difference to a threshold; and
if the difference exceeds the threshold, generating an alert for presentation to the user and/or for transmitting to a remote electronic device.
